(19) United States
(12) Patent Application Publication
(10) Pub. No.: US 2014/0096485 A1
(43) Pub. Date: Apr. 10, 2014

(54) MEDICINAL TEA

(71) Applicant: ABEER ABDUL AZIZ KHALAF MALALLAH, AL-QURAIN (KW)

(72) Inventor: ABEER ABDUL AZIZ KHALAF MALALLAH, AL-QURAIN (KW)

(21) Appl. No.: 13/647,287

(22) Filed: Oct. 8, 2012

(51) Int. Cl. A61K 36/74; B65B 9/00; A61P 11/00; A61P 15/02; A61P 1/00

(52) U.S. Cl. 53/435; 424/739

(57) ABSTRACT

The medicinal tea includes a natural, herbal composition for treating female menstrual pains and post-delivery symptoms. The composition eliminates the side effects of common pain relief or sedative remedies. The composition includes Salvia officinalis, cumin, Artemisia herba-alba, Nigella sativa, Pimpinella anisum, coffee husk, Carum copticom, cinnamon, Achillea fragrantissima, and mint in specific amounts which together provide diuretic, sedative, menstrual and digestive benefits. This composition is easily digestible and provides immediate benefits without any harmful side effects.

### BACKGROUND OF THE INVENTION

1. Field of the Invention

The present invention relates to herbal remedies, and particularly to a medicinal tea that is believed to provide menstrual and other health benefits.

2. Description of the Related Art

Many women of physical maturity suffer menstrual cramps and related symptoms on a monthly basis. There exist several medications such as pain relievers and sedatives that combat this condition. However, most of these remedies are composed of processed chemicals. As such, while they provide temporary relief, they have side effects such as chronic headache, listlessness, nausea and vomiting, Some can also cause liquidity in the blood when used too often.

Studies have found that arbitrary intake of sedatives can cause infection of the bottom of the esophagus and the stomach lining which can lead to ulcers and duodenum as well as infections to the kidney and liver. Certain sedatives can also cause blood fluidity. Intake of these types of sedatives or pain relievers without proper physician consultation can lead to complications, especially for those suffering from hypertension. Moreover, it has been shown that improper intake of these sedatives can cause liver and kidney failures in addition to skin irritations or allergies in persons who suffer chest allergy or asthma.

In light of the above, it would be a benefit in the art of medicines to provide a remedy formed from natural ingredients that can relieve menstrual cramps and related symptoms without harmful side effects. Thus, a medicinal tea solving the aforementioned problems is desired.

### SUMMARY OF THE INVENTION

The medicinal tea includes a natural, herbal composition for treating female menstrual pains and post-delivery symptoms. The composition eliminates the side effects of common pain relief or sedative remedies. The composition includes salvia officinalis, cumin, artemesia herba alba, nigella saliva, pimpinella anisum, coffee husk, carum copticum, cinnamon, achillea fragrantissima, and mint in specific amounts, which together provide diuretic, sedative, menstrual and digestive benefits. This composition is easily digestible and provides immediate benefits without any harmful side effects.

These and other features of the present invention will become readily apparent upon further review of the following specification.

### DETAILED DESCRIPTION OF THE PREFERRED EMBODIMENTS

The medicinal tea includes several natural herbs to provide protection and relief of pain that a woman suffers before, during and after menstruation, in addition to certain instances of stomach disorders, toxicity, constipation, and cold. The medicinal tea includes the following ingredients listed in Table 1 in the amounts listed thereon, the measured amounts being in grams (g). While each ingredient provides independent health benefits, the combination of these ingredients has been found to be especially effective in treating menstrual pain, as well as some digestive and cold related symptoms.

TABLE 1
Table 1: Medicinal Tea Composition

| Ingredients | Amount (g) |
|---|---|
| Salvia officinalis | 60 |
| Cumin | 20 |
| Artemisia herba-alba | 20 |
| Nigella Sativa | 25 |
| Pimpinella anisum | 20 |
| Coffee Husk | 25 |
| Carum copticom | 20 |
| Cinnamon | 25 |
| Achillea fragrantissima | 20 |
| Mint | 10 |

The medicinal tea is first prepared by cleaning the Salvia officinalis to remove impurities. The cleaned Salvia officinalis is ground and mixed with cumin and Artemisia herba-alba. Salvia officinalis helps clean the intestines and provides other gastro intestinal benefits, such as relief of abdominal pains and prevention of regurgitation, diarrhea and dyspepsia. Salvia officinalis has also been found to help regulate menstruation and other female-related fluid secretions, fight gingivitis, and relieve sore throats. Cumin also provides gastrointestinal benefits such as an antiparasitic agent against intestinal worms, gas and dyspepsia prevention. Artemisia herba-alba also acts as an antiparasitic agent for worms. It contains santonin, an anthelmintic substance. Moreover, Artemisia herba-alba improves menstrual regulation, urine secretion and relieves abdominal pain.

Next, the Nigella saliva is cleaned and roasted until dark brown in color. The roasted Nigella saliva is cooled and added to the mixture. Nigella saliva has several respiratory and gastrointestinal benefits. It helps relieve cold symptoms such as coughs and nasal congestion, asthma, dyspepsia, and stomach gases. It also helps relieve common pains such as headaches and toothaches. Nigella saliva also provides several benefits for women, such as improved urine secretion, remedy menstrual disorders, enhance milk production, energizes the body and fight fatigue.

The Pimpinella anisum is coarsely ground prior to adding the same to the mixture. A coarse grind is desired in order to obtain the full benefits of this herb. A finer grind will result in less effectiveness. Pimpinella anisum contains anethole, a phytoestrogen. This substance has the ability to cause estrogenic and/or antiestrogenic effects. Thus, Pimpinella anisum assists in controlling menstrual conditions.

Then the coffee husk requires slight toasting to allow the oils to bloom. After cooling, the lightly toasted coffee husk is added to the above ingredients. Coffee husk has been found to be beneficial in maintaining the health of the uterus, remove impurities and purify blood.

After the coffee husk has been added, the Carum copticom, cinnamon and Achillea fragrantissima are ground and added to the mixture. The Carum copticom acts as a digestive aid, while cinnamon increases the body's defenses against cold and influenza. Cinnamon also enhances blood circulation and breathing, generates better secretion, improves vessel health, stimulates the intestines, and assists in cleaning and relieving the digestive system. The Achillea fragrantissima energizes the body and strengthens the body's immune system. Moreover, it also acts as an anthelmintic and relieves chronic diarrhea. This herb also promotes menstruation.

Fresh mint is placed on a flat plate with filter paper and sun dried. The dried mint is then ground and added to the rest of the mixture. Mint is a digestive aid for after meals. It also has many benefits against cold and influenza attacks because it helps dilate the air passages and relieves nasal congestion.

About a teaspoon of the mixed ingredients is placed on filter paper and sealed to construct discrete tea packets or teabags. It is recommended that one or two teabags be used to brew the medicinal tea. The tea should steep for about fifteen minutes in order to fully extract all the herbal benefits. A regimen of one or two daily cups has been found to be beneficial, especially after meals. For women, it has been found that the medicinal tea helps relieve menstrual pain and regulate fluids and secretions stemming from post-delivery symptoms. In addition to the above, the medicinal tea removes toxins; relieves indigestion, constipation and excessive gas; fights stomach worms; energizes the body and improves immunity. Thus, it can be seen that the medicinal tea is a very natural herbal remedy easily digested for body efficacy.

It is to be understood that the present invention is not limited to the embodiments described above, but encompasses any and all embodiments within the scope of the following claims.

I claim:

1. A medicinal tea, comprising an herbal composition comprising a mixture of Salvia officinalis, cumin, Artemesia herba-alba, Nigella sativa, Pimpinella anisum, coffee husk, Carum copticum, cinnamon, Achillea fragrantissima, and mint.

2. The medicinal tea according to claim 1, wherein said composition comprises about 60 g Salvia officinalis, about 20 g cumin, about 20 g Artemesia herba-alba, about 25 g Nigella sativa, about 20 g Pimpinella anisum, about 25 g coffee husk, about 20 g Carum copticum, about 25 g cinnamon, about 20 g Achillea fragrantissima, and about 10 g mint.

3. A process of making medicinal tea for treating menstrual, digestive and respiratory symptoms comprising the steps of:
providing given amounts of Salvia officinalis, cumin, Artemesia herba-alba, Nigella saliva, Pimpinella anisum, coffee husk, Carum copticum, cinnamon, Achillea fragrantissima, and mint;
grinding the Salvia officinalis, Pimpinella anisum, Carum copticum, cinnamon, Achillea fragrantissima, and mint;
roasting the Nigella saliva;
toasting the coffee husk;
mixing the Salvia officinalis, cumin, Artemesia herba-alba, Nigella saliva, Pimpinella anisum, coffee husk, Carum copticum, cinnamon, Achillea fragrantissima, and mint thereby forming a tea composition;
providing at least one sheet of filter paper;
placing at least one given amount of the tea composition onto the filter paper;
placing another sheet of filter paper over the at least one sheet of filter paper;
sealing both sheets of filter paper to form at least one discrete teabag filled with the given amount of tea composition; and
cutting the sealed filter paper into individual teabags.

4. The process of making medicinal tea for treating menstrual, digestive and respiratory symptoms according to claim 3, further comprising the step of cleaning said Salvia officinalis prior to said grinding step.

5. The process of making medicinal tea for treating menstrual, digestive and respiratory symptoms according to claim 3, wherein said step of roasting said Nigella sativa comprises the steps of:
cleaning said Nigella sativa prior to roasting; and
roasting said cleaned Nigella sativa until dark brown in color.

6. The process of making medicinal tea for treating menstrual, digestive and respiratory symptoms according to claim 3, wherein said step of grinding said Pimpinella anisum comprises the step of coarsely grinding said Pimpinella anisum.

7. The process of making medicinal tea for treating menstrual, digestive and respiratory symptoms according to claim 3, wherein said step of toasting coffee husk comprises the steps of:
lightly toasting said coffee husk to facilitate blooming of said coffee husk; and
cooling said lightly toasted coffee husk.

8. The process of making medicinal tea for treating menstrual, digestive and respiratory symptoms according to claim 3, wherein said step of grinding said mint comprises the steps of:
providing fresh mint; and
sun drying said fresh mint prior to grinding said mint.
